(19) United States
(12) Patent Application Publication
(10) Pub. No.: US 2022/0280720 A1
(43) Pub. Date: Sep. 8, 2022

(54) METHOD AND SYSTEM FOR DETERMINING GLUCOSE CHANGE IN A SUBJECT

(71) Applicant: Eli Lilly and Company, Indianapolis, IN (US)

(72) Inventors: Anas EL FATHI, Lasalle, Québec (CA); Ahmad HAIDAR, Montréal, Québec (CA)

(21) Appl. No.: 17/625,180

(22) PCT Filed: Jul. 9, 2020
(86) PCT No.: PCT/IB2020/056467

§ 371 Date: Jan. 6, 2022

Related U.S. Application Data
(60) Provisional application No. 62/871,931, filed on Jul. 9, 2019.

(51) Int. Cl. A61M 5/172; G16H 40/63; G16H 50/20; A61B 5/00; G16H 20/17; A61B 5/145

(52) U.S. Cl. CPC A61M 5/1723; G16H 40/63; G16H 50/20; A61B 5/725; A61B 5/7275; G16H 20/17; A61B 5/14532; A61M 2230/201

(57) ABSTRACT

There is provided a method and a system for determining glucose change in a subject, which includes receiving subject model parameters. The subject model parameters of a state-based model of the subject may have been estimated based on: actual glucose measurements and past subject model parameters. An innovation parameter and an innovation covariance parameter are determined using a Kalman filter based on the subject model parameters and a previous state of the subject. A test statistic is calculated based on the determined innovation parameter and the innovation covariance parameter. The calculated test statistic is compared to a given threshold. In response to the calculated test statistic being above the given threshold, an indication of the glucose change is outputted.

### CROSS-REFERENCE TO RELATED APPLICATIONS

The present application claims the benefit of U.S. Provisional Patent Application No. 62/871,931, filed Jul. 9, 2019, which is incorporated by reference herein in its entirety.

### FIELD

The present technology relates to drug monitoring systems in general and more specifically to a method of and a system for determining if a glucose change of a diabetic subject is anomalous or indicative of an issue, for example if a consumed meal has not been logged by the subject in an artificial pancreas system.

### BACKGROUND

In healthy individuals, plasma glucose concentration is tightly regulated by the action of the hormones secreted by the endocrine pancreas, principally, insulin and glucagon. Insulin is secreted by the pancreatic beta cells to signal organs to absorb glucose and glucagon is secreted by the pancreatic alpha cells to signal the liver to produce glucose. In type 1 diabetes, insulin secretion is lost due to the autoimmune destruction of the beta cells.

Type 1 diabetes is currently treated with life-long insulin-replacement therapy implemented using multiple daily injections (MDI), or by continuous subcutaneous (under the skin tissue) insulin infusion (CSII) via a portable pump. Both therapies follow a basal-bolus insulin injection pattern which aims to mimic the physiological plasma insulin secretion seen in healthy individuals. Basal insulin stands for insulin needs that keep a constant glucose level under fasting conditions and insulin boluses are doses of insulin usually given to cover the expected glucose increase from consumed meals.

Tight glucose control is key for type 1 diabetes patients. Sustained elevation of glucose levels (hyperglycemia) leads to long-term complications such as heart disease, blindness, kidney failure, and lower-extremity amputations. Low glucose levels (hypoglycemia) are a limiting factor of glycemic control, since non-severe hypoglycemia may lead to anxiety, nausea, confusion, blurred vision, and difficulty in speaking, while severe hypoglycemia leads to coma or seizure and necessitates assistance. A target of HbA1c (a biomarker correlated with the mean blood glucose level over a period of three months) below 7.0% is recommended for most patients with type 1 diabetes.

Despite advances in insulin analogs, insulin pumps, and continuous glucose sensors, most patients do not achieve acceptable glucose targets. Advances in glucose sensors have motivated the development of the artificial pancreas (AP), a closed-loop insulin delivery system that automatically regulates glucose levels in patients with type 1 diabetes. In the artificial pancreas, a control procedure adjusts the pump insulin infusion rate based on continuous glucose sensor readings. Artificial pancreas systems are considered the most promising therapy for type 1 diabetes. Attempts for a fully automated closed-loop insulin delivery system have been investigated, yet, the most prevailing artificial pancreas systems still rely on the user prompt to provide meal-accompanying insulin bolus.

In conventional insulin therapy, a primary factor for poor glucose control in adolescents is the omission of insulin bolus delivery at mealtimes. It has been observed that 65% of adolescents missed one or more mealtime bolus per week, which was associated with a significantly higher HbA1c compared to adolescents that missed less than one bolus per week (8.8% and 8.0% respectively). Another study observed that over a third of adolescents missed more than 15% of their required boluses. Similar to conventional insulin therapy, the performance of a closed-loop (CL) insulin delivery may also be affected after a missed bolus. The addition of a meal detection module which will detect an unknown meal to the artificial pancreas system and signal the infusion of more insulin may improve the performance of the artificial pancreas.

In the artificial pancreas system when an unknown meal is consumed, the closed-loop feedback mechanism reacts to glucose level changes by altering the pump's insulin basal rate. Generally, a significant amount of insulin is needed to cover the glucose increase from meals, up to 20% of the patient total daily insulin dose in some cases. As a result, without delivering an insulin bolus, the artificial pancreas is unable of providing the needed amount of insulin in a short period of time. Thus, hyperglycemic events with unwanted high glucose levels become unavoidable. Furthermore, if the feedback reacts aggressively by infusing a large amount of insulin to prevent glucose from further increasing, then late post-meal hypoglycemia may occur due to the slow absorption of insulin delivery (since the delivered insulin continues to act beyond meal absorption). A distinct strategy is needed to mitigate hyperglycemia and hypoglycemia after a missed bolus.

### SUMMARY

It is an object of the present technology to ameliorate at least some of the inconveniences present in the prior art. One or more embodiments of the present technology may provide and/or broaden the scope of approaches to and/or methods of achieving the aims and objects of the present technology.

One or more embodiments of the present technology have been developed based on inventors' appreciation that glucose control is degraded significantly after a missed prandial bolus. The performance of closed-loop delivery system after missed boluses could be improved if a control algorithm is augmented with a meal detection technique.

Inventors' have appreciated that that automatically detecting the meal (which had no bolus delivered) and notify a diabetic subject could improve a quality of life and health of a diabetic user. In one non-limiting example, the system could notify the user, which can take an action, such as deliver the forgotten insulin to himself or herself. In another non-limiting example, users of conventional pump therapy or multiple daily injections could be reminded if they eat a meal and forget to provide a bolus.

Such a system could be used to detect disturbances that raise glucose values, such as infusion set failure, or missed meals.

Inventors have also appreciated that such a technology could be used online or offline, to analyze and model data, verify algorithm performance, and as a non-limiting example to identify unannounced meals and hypoglycemia treatment.

Thus, one or more embodiments of the present technology are directed to a method and a system for detecting a glucose change in a subject.

In accordance with a broad aspect of the present technology, there is provided a computer-implemented method for determining a glucose change in a subject, the method is executable by an electronic device. the method comprises: receiving subject model parameters of a state-based model of the subject, determining, using a Kalman filter, an innovation parameter and an innovation covariance parameter based on the subject model parameters and a previous state of the subject, calculating a test statistic based on the determined innovation parameter and the innovation covariance parameter, comparing the calculated test statistic to a given threshold, and in response to the calculated test statistic is above the given threshold, outputting an indication of the glucose change.

In one or more embodiments of the method, the method further includes, prior to said receiving the subject model parameters: receiving, by the electronic device, actual glucose measurements of the subject, and receiving past subject model parameters, and said receiving the subject model parameters of a state-based model of the subject comprises estimating the subject model parameters based on: the actual glucose measurements, and the past subject model parameters

In one or more embodiments of the method, the method further comprises transmitting the indication to at least one of: a display-interface of the electronic device and an artificial pancreas system of the subject.

In one or more embodiments of the method, the test statistic is above the given threshold is indicative of the Kalman filter is inconsistent.

In one or more embodiments of the method, said estimating the subject model parameters comprises using a maximum posteriori probability (MAP) estimate.

In one or more embodiments of the method, said estimating the subject model parameters is further based on: previous glucose measurements, previous insulin measurements and previous consumed meals.

In one or more embodiments of the method, the test statistic is above the given threshold is indicative of the innovation parameter not is: independent and identically distributed with a zero-mean Gaussian distribution with a covariance corresponding to the covariance of the innovation parameter.

In one or more embodiments of the method, the glucose change is indicative of an unknown meal, the unknown meal not having been logged by the subject.

In one or more embodiments of the method, the given threshold is based on a predetermined number of false positives.

In one or more embodiments of the method, the method further comprises, prior to said receiving the past subject model parameters: initializing the past subject model parameters based on: a daily total dose, a basal insulin, and a carbohydrate ratio of the subject.

In one or more embodiments of the method, the actual glucose measurements are received from a glucose sensor connected to the electronic device.

In one or more embodiments of the method, the method further, prior to said transmitting the indication to the at least one of: the display-interface of the electronic device and the artificial pancreas system of the subject: determining an insulin bolus of the unknown meal not having been logged by the given user based on: a remaining meal, a patient carbohydrate ratio and a glucose level, and said transmitting the indication comprises transmitting the insulin bolus.

In one or more embodiments of the method, the method further comprises, prior to said determining the insulin bolus: determining, based on the innovation parameter and the innovation covariance parameter, an unknown meal amount and an unknown meal time.

In one or more embodiments of the method, the calculated test statistic is representative of a cumulative sum of a correlation between the innovation parameter and a glucose change based on the unknown meal amount and the unknown meal time weighted by the innovation covariance parameter.

In one or more embodiments of the method, the given threshold is determined based on a: given false positive rate for a random variable with a zero-mean Gaussian distribution and covariance proportional to the square of a most probable glucose increase due to a most probable meal amount and meal time weighted by the innovation covariance parameter.

In accordance with a broad aspect of the present technology, there is provided a computer-implemented method for detecting meals consumed by a patient, the method being executed by a processor, the method comprises determining a mismatch between actual glucose measurements and predicted glucose measurements, determining a probability that a meal has been consumed based at least in part on the determined mismatch, and in response to the determined probability, determining a medication bolus.

In one or more embodiments of the method, said determining the probability that a meal has been consumed is based, at least in part, on an actual glucose level, a target glucose level, and insulin-on-board.

In one or more embodiments of the method, the method further comprises estimating a meal size and a time of consumption of the meal.

In one or more embodiments of the method, said determining the medication bolus is based, at least in part, on at least one of: the estimated meal size and the estimated time of consumption of the meal.

In one or more embodiments of the method, said determining that a meal has been consumed is in response to the determined probability breaching a threshold.

In accordance with a broad aspect of the present technology, there is provided a system for determining a glucose change in a subject. the system comprises: a processor, a non-transitory storage medium operatively connected to the processor, the storage medium includes computer-readable instructions, the processor, upon executing the computer-readable instructions, is configured for: receiving subject model parameters of a state-based model of the subject, determining, using a Kalman filter, an innovation parameter and an innovation covariance parameter based on the subject model parameters and a previous state of the subject, calculating a test statistic based on the determined innovation parameter and the innovation covariance parameter, comparing the calculated test statistic to a given threshold, and in response to the calculated test statistic is above the given threshold, outputting an indication of the glucose change.

In one or more embodiments of the system, the processor is further configured for, prior to said receiving the subject model parameters: receiving actual glucose measurements of the subject, and receiving past subject model parameters, and said receiving the subject model parameters of a state-based model of the subject comprises estimating the subject model parameters based on: the actual glucose measurements and the past subject model parameters

In one or more embodiments of the system, the processor is further configured for transmitting the indication to at least one of: a display-interface operatively connected to the processor, and an artificial pancreas system of the subject.

In one or more embodiments of the system, the test statistic is above the given threshold is indicative of the Kalman filter is inconsistent.

In one or more embodiments of the system, said estimating the subject model parameters comprises using a maximum posteriori probability (MAP) estimate.

In one or more embodiments of the system, said estimating is further based on: previous glucose measurements, previous insulin measurements and previous consumed meals.

In one or more embodiments of the system, the test statistic is above the given threshold is indicative of the innovation parameter not is: independent and identically distributed with a zero-mean Gaussian distribution with a covariance corresponding to the covariance of the innovation parameter.

In one or more embodiments of the system, the glucose change is indicative of an unknown meal, the unknown meal not having been logged by the subject.

In one or more embodiments of the system, the given threshold is based on a predetermined number of false positives.

In one or more embodiments of the system, the processor is further configured for, prior to said receiving the past subject model parameters: initializing the past subject model parameters based on: a daily total dose, a basal insulin and a carbohydrate ratio of the subject.

In one or more embodiments of the system, the actual glucose measurements are received from a glucose sensor connected to the processor.

In one or more embodiments of the system, the processor is further configured for, prior to said transmitting the indication to the at least one of: the display-interface operatively connected to the processor and the artificial pancreas system of the subject: determining an insulin bolus of the unknown meal not having been logged by the given user based on: a remaining meal, a patient carbohydrate ratio and a glucose level, and said transmitting the indication comprises transmitting the insulin bolus.

In one or more embodiments of the system, the processor is further configured for, prior to said determining the insulin bolus: determining, based on the innovation parameter and the innovation covariance parameter, an unknown meal amount and an unknown meal time.

In one or more embodiments of the system, the test statistic is representative of a cumulative sum of a correlation between the innovation parameter and a glucose change based on the unknown meal amount and the unknown meal time weighted by the innovation covariance parameter.

In one or more embodiments of the system, the given threshold is determined based on a: given false positive rate for a random variable with a zero-mean Gaussian distribution and covariance proportional to the square of a most probable glucose increase due to a most probable meal amount and meal time weighted by the innovation covariance parameter.

In accordance with another broad aspect, there is provided a computer-implemented method for detecting meals consumed by a patient. The method comprises determining a mismatch between actual glucose measurements and predicted glucose measurements. Based at least in part on the determined mismatch, the method comprises determining a probability that a meal has been consumed. In response to the determined probability, the method comprises determining a medication bolus.

In one embodiment of the method, the probability that a meal has been consumed is based, at least in part, on an actual glucose level, a target glucose level, and insulin-on-board.

In one embodiment of the method, the method further comprises estimating a meal size and a time of consumption of the meal.

In one embodiment of the method, an amount of the medication bolus is based, at least in part, on the estimated meal size and/or the estimated time of consumption of the meal.

In one embodiment of the method, the method further comprises determining that a meal has been consumed in response to the determined probability breaching a threshold.

In accordance with another broad aspect, there is provided a system for detecting meals consumed by a patient. The system comprises: a processor and a non-transitory storage medium operatively connected to the processor, the storage medium comprises computer-readable instructions, the processor, upon executing the computer-readable instructions, is configured for: determining a mismatch between actual glucose measurements and predicted glucose measurements, determining a probability that a meal has been consumed based at least in part on the determined mismatch, and in response to the determined probability, determining a medication bolus.

In one or more embodiments of the system, said determining the probability that a meal has been consumed is based, at least in part, on an actual glucose level, a target glucose level, and insulin-on-board.

In one or more embodiments of the system, the method further comprises estimating a meal size and a time of consumption of the meal.

In one or more embodiments of the system, said determining the medication bolus is based, at least in part, on at least one of: the estimated meal size and the estimated time of consumption of the meal.

In one or more embodiments of the system, said determining that a meal has been consumed is in response to the determined probability breaching a threshold.

In the context of the present specification, “electronic device” is any computing apparatus or computer hardware that is capable of running software appropriate to the relevant task at hand. Thus, some (non-limiting) examples of electronic devices include general purpose personal computers (desktops, laptops, netbooks, etc.), mobile computing devices, smartphones, and tablets, and network equipment such as routers, switches, and gateways. It should be noted that an electronic device in the present context is not precluded from acting as a server to other electronic devices. The use of the expression “an electronic device” does not preclude multiple electronic devices being used in receiving/sending, carrying out or causing to be carried out any task or request, or the consequences of any task or request, or steps of any method described herein. In the context of the present specification, a “client device” refers to any of a range of end-user client electronic devices, associated with a user, such as personal computers, tablets, smartphones, and the like.

In the context of the present specification, the expression “computer readable storage medium” (also referred to as “storage medium” and “storage”) is intended to include non-transitory media of any nature and kind whatsoever, including without limitation RAM, ROM, disks (CD-ROMs, DVDs, floppy disks, hard drivers, etc.), USB keys, solid state-drives, tape drives, etc. A plurality of components may be combined to form the computer information storage media, including two or more media components of a same type and/or two or more media components of different types.

In the context of the present specification, a “database” is any structured collection of data, irrespective of its particular structure, the database management software, or the computer hardware on which the data is stored, implemented or otherwise rendered available for use. A database may reside on the same hardware as the process that stores or makes use of the information stored in the database or it may reside on separate hardware, such as a dedicated server or plurality of servers.

In the context of the present specification, the expression “information” includes information of any nature or kind whatsoever capable of being stored in a database. The information includes, but is not limited to audiovisual works (images, movies, sound records, presentations etc.), data (location data, numerical data, etc.), text (opinions, comments, questions, messages, etc.), documents, spreadsheets, lists of words, etc.

In the context of the present specification, unless expressly provided otherwise, an “indication” of an information element may be the information element itself or a pointer, reference, link, or other indirect mechanism enabling the recipient of the indication to locate a network, memory, database, or other computer-readable medium location from which the information element may be retrieved. For example, an indication of a document could include the document itself (i.e. its contents), or it could be a unique document descriptor identifying a file with respect to a particular file system, or some other means of directing the recipient of the indication to a network location, memory address, database table, or other location where the file may be accessed. As one skilled in the art would recognize, the degree of precision required in such an indication depends on the extent of any prior understanding about the interpretation to be given to information being exchanged as between the sender and the recipient of the indication. For example, if it is understood prior to a communication between a sender and a recipient that an indication of an information element will take the form of a database key for an entry in a particular table of a predetermined database containing the information element, then the sending of the database key is all that is required to effectively convey the information element to the recipient, even though the information element itself was not transmitted as between the sender and the recipient of the indication.

In the context of the present specification, the expression “communication network” is intended to include a telecommunications network such as a computer network, the Internet, a telephone network, a Telex network, a TCP/IP data network (e.g., a WAN network, a LAN network, etc.), and the like. The term “communication network” includes a wired network or direct-wired connection, and wireless media such as acoustic, radio frequency (RF), infrared and other wireless media, as well as combinations of any of the above.

In the context of the present specification, the words “first”, “second”, “third”, etc. have been used as adjectives only for the purpose of allowing for distinction between the nouns that they modify from one another, and not for the purpose of describing any particular relationship between those nouns. Thus, for example, it should be understood that, the use of the terms “first server” and “third server” is not intended to imply any particular order, type, chronology, hierarchy or ranking (for example) of/between the server, nor is their use (by itself) intended imply that any “second server” must necessarily exist in any given situation. Further, as is discussed herein in other contexts, reference to a “first” element and a “second” element does not preclude the two elements from being the same actual real-world element. Thus, for example, in some instances, a “first” server and a “second” server may be the same software and/or hardware, in other cases they may be different software and/or hardware.

Implementations of the present technology each have at least one of the above-mentioned object and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects and advantages of implementations of the present technology will become apparent from the following description, the accompanying drawings and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 depicts a schematic diagram of an electronic device in accordance with non-limiting embodiments of the present technology.

FIG. 2 depicts a schematic diagram of a system in accordance with non-limiting embodiments of the present technology.

FIG. 3 depicts a schematic diagram of an unknown meal detection procedure in accordance with non-limiting embodiments of the present technology.

FIG. 4 depicts a block diagram of a flowchart of a method of determining a glucose change in a subject, the method being executed in accordance with non-limiting embodiments of the present technology.

FIG. 5A depicts an exemplary plot of results of a sample simulation, the meal detection procedure detects an announced meal and provides a bolus of 2U. Due to the model's variability, glucose levels often increase or decrease without an apparent reason, which makes it challenging for the meal detection procedure.

FIG. 5B depicts an exemplary plot of simulations where a false positive(FP) occurred, where a meal is flagged at 15:30 after 3.5 hours of having the lunch meal and where the algorithm provides a bolus of 1.8U and no hypoglycemia is observed for the next 4.5 hours.

FIG. 6 depicts an exemplary plot of percentage time (relative to the 8 hours after the lunch meal) spent in hypoglycemia and hyperglycemia for the three conducted experiments (n=1536), where CL+B corresponds to no meal detection and with the lunch announced and bloused, where CL+MD corresponds to use of a meal detection procedure, with the lunch not announced, and where CL corresponds to no meal detection with the lunch not announced.

FIG. 7 depicts an exemplary plot of clinical data showing the meal detection procedure performance, where an unknown meal of 60 g was consumed at 13:00, and the meal was detected at 13:40, and where a bolus of 0.9U was delivered.

FIG. 8 depicts an exemplary plot of incremental glucose after consuming a meal without bolus for four patients using conventional pump therapy, closed-loop or closed-loop with a meal detection, where the diamonds indicate when a correction bolus was delivered either for safety reasons or automatically by the meal detection procedure.

### DETAILED DESCRIPTION

The examples and conditional language recited herein are principally intended to aid the reader in understanding the principles of the present technology and not to limit its scope to such specifically recited examples and conditions. It will be appreciated that those skilled in the art may devise various arrangements which, although not explicitly described or shown herein, nonetheless embody the principles of the present technology and are included within its spirit and scope.

Furthermore, as an aid to understanding, the following description may describe relatively simplified implementations of the present technology. As persons skilled in the art would understand, various implementations of the present technology may be of a greater complexity.

In some cases, what are believed to be helpful examples of modifications to the present technology may also be set forth. This is done merely as an aid to understanding, and, again, not to define the scope or set forth the bounds of the present technology. These modifications are not an exhaustive list, and a person skilled in the art may make other modifications while nonetheless remaining within the scope of the present technology. Further, where no examples of modifications have been set forth, it should not be interpreted that no modifications are possible and/or that what is described is the sole manner of implementing that element of the present technology.

Moreover, all statements herein reciting principles, aspects, and implementations of the present technology, as well as specific examples thereof, are intended to encompass both structural and functional equivalents thereof, whether they are currently known or developed in the future. Thus, for example, it will be appreciated by those skilled in the art that any block diagrams herein represent conceptual views of illustrative circuitry embodying the principles of the present technology Similarly, it will be appreciated that any flowcharts, flow diagrams, state transition diagrams, pseudo-code, and the like represent various processes which may be substantially represented in computer-readable media and so executed by a computer or processor, whether or not such computer or processor is explicitly shown.

The functions of the various elements shown in the figures, including any functional block labeled as a “processor” or a “graphics processing unit”, may be provided through the use of dedicated hardware as well as hardware capable of executing software in association with appropriate software. When provided by a processor, the functions may be provided by a single dedicated processor, by a single shared processor, or by a plurality of individual processors, some of which may be shared. In some non-limiting embodiments of the present technology, the processor may be a general purpose processor, such as a central processing unit (CPU) or a processor dedicated to a specific purpose, such as a graphics processing unit (GPU). Moreover, explicit use of the term “processor” or “controller” should not be construed to refer exclusively to hardware capable of executing software, and may implicitly include, without limitation, digital signal processor (DSP) hardware, network processor, application specific integrated circuit (ASIC), field programmable gate array (FPGA), read-only memory (ROM) for storing software, random access memory (RAM), and non-volatile storage. Other hardware, conventional and/or custom, may also be included.

Software modules, or simply modules which are implied to be software, may be represented herein as any combination of flowchart elements or other elements indicating performance of process steps and/or textual description. Such modules may be executed by hardware that is expressly or implicitly shown.

With these fundamentals in place, some non-limiting examples to illustrate various implementations of aspects of the present technology will be considered.

### Electronic Device

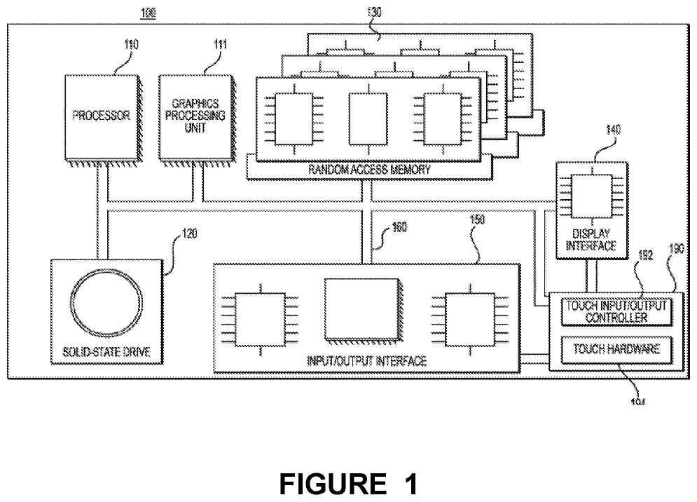

Referring to FIG. 1, there is shown an electronic device 100 suitable for use with some implementations of the present technology, the electronic device 100 comprising various hardware components including one or more single or multi-core processors collectively represented by processor 110, a graphics processing unit (GPU) 111, a solid-state drive 120, a random-access memory 130, a display interface 140, and an input/output interface 150.

Communication between the various components of the electronic device 100 may be enabled by one or more internal and/or external buses 160 (e.g. a PCI bus, universal serial bus, IEEE 1394 “Firewire” bus, SCSI bus, Serial-ATA bus, etc.), to which the various hardware components are electronically coupled.

The input/output interface 150 may be coupled to a touchscreen 190 and/or to the one or more internal and/or external buses 160. The touchscreen 190 may be part of the display. In some embodiments, the touchscreen 190 is the display. The touchscreen 190 may equally be referred to as a screen 190. In the embodiments illustrated in FIG. 1, the touchscreen 190 comprises touch hardware 194 (e.g., pressure-sensitive cells embedded in a layer of a display allowing detection of a physical interaction between a user and the display) and a touch input/output controller 192 allowing communication with the display interface 140 and/or the one or more internal and/or external buses 160. In some embodiments, the input/output interface 150 may be connected to a keyboard (not shown), a mouse (not shown) or a trackpad (not shown) allowing the user to interact with the electronic device 100 in addition or in replacement of the touchscreen 190.

According to implementations of the present technology, the solid-state drive 120 stores program instructions suitable for being loaded into the random access memory 130 and executed by the processor 110 and/or the GPU 111 for determining whether a diabetic subject has consumed a meal. For example, the program instructions may be part of a library or an application.

The electronic device 100 may be a server, a desktop computer, a laptop computer, a tablet, a smartphone, a personal digital assistant or any device that may be configured to implement the present technology, as it may be understood by a person skilled in the art.

### System

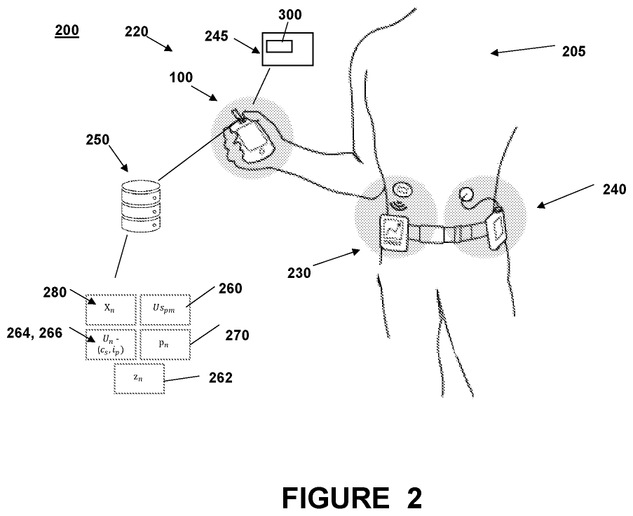

Referring to FIG. 2, there is shown a schematic diagram of a system 200, the system 200 being suitable for implementing non-limiting embodiments of the present technology. It is to be expressly understood that the system 200 as depicted is merely an illustrative implementation of the present technology. Thus, the description thereof that follows is intended to be only a description of illustrative examples of the present technology. This description is not intended to define the scope or set forth the bounds of the present technology. In some cases, what are believed to be helpful examples of modifications to the system 200 may also be set forth below. This is done merely as an aid to understanding, and, again, not to define the scope or set forth the bounds of the present technology. These modifications are not an exhaustive list, and, as a person skilled in the art would understand, other modifications are likely possible. Further, where this has not been done (i.e., where no examples of modifications have been set forth), it should not be interpreted that no modifications are possible and/or that what is described is the sole manner of implementing that element of the present technology. As a person skilled in the art would understand, this is likely not the case. In addition, it is to be understood that the system 200 may provide in certain instances simple implementations of the present technology, and that where such is the case they have been presented in this manner as an aid to understanding. As persons skilled in the art would understand, various implementations of the present technology may be of a greater complexity.

The system 200 comprises inter alia the electronic device 100, a database 250, and an artificial pancreas system 220.

The system 200 is associated with a diabetic subject 205 or diabetic user 205.

The electronic device 100 is associated with a diabetic user 205. As a non-limiting example, the electronic device 100 may be a smartphone of the diabetic user 205. The diabetic user 205 may input information relating to his health and diabetes into the electronic device 100, which stores the information into the database 250. In one embodiment, the electronic device 100 may be part of the artificial pancreas system (e.g. in a component of the artificial pancreas system 220). In an alternative embodiment, the electronic device 100 may be a desktop computer of the diabetic user 205.

The electronic device 100 is configured to inter alia: (i) model the glucoregulatory system of the diabetic user 205; (ii) predict glucose measurements; (iii) determine, based on the predilected measurements, if an insulin bolus has been missed due to a meal consumed by the user 205 not having been logged into the electronic device 100; and (iv) transmit information to the artificial pancreas system 220 for insulin delivery to the user 205. How the electronic device 100 is configured to achieve that purpose will be explained in more detail hereinbelow.

The artificial pancreas system 220, also known as closed-loop system, an automated insulin delivery system or an autonomous system for glycemic control, is configured to mimic a glucose regulating function of a healthy pancreas. The artificial pancreas system 220 is operatively connected to and associated with the diabetic user 205.

The artificial pancreas system 220 comprises: a continuous glucose monitoring system (CGM) 230, an insulin infusion pump 240, and a control procedure 245.

The CGM system 230 provides a steady stream of information that reflects the user's 205 blood glucose levels. The CGM 230 comprises a sensor placed subcutaneously under the patient's skin (not depicted) which measures the glucose in the fluid around the cells (interstitial fluid) which is associated with blood glucose levels. The CGM system 230 may have a user interface such as a screen or touchscreen (not depicted) and/or may transmit the glucose related information to the electronic device 100 of the user 205 or another electronic device (not depicted) via a communication link (not numbered) over a communication network (not depicted).

In one embodiment, the glucose monitoring system 230 transmits information reflecting the user's 205 blood glucose levels for storage in the database 250.

In one embodiment, the electronic device 100 executes the control procedure 245 which receives information from the CGM 230 and performs a series of mathematical calculations. Based on these calculations, the electronic device 100 sends dosing instructions to the infusion pump. In an alternative embodiment, the control procedure 245 can be executed on any number of devices including the insulin infusion pump 240, such as but not limited to a desktop computer, a remote server, and a smartphone.

The control procedure 245 includes a meal detection procedure 300, which will be explained in more detail below.

The insulin infusion pump 240 adjusts the insulin delivery based on the instructions received from the control procedure 245.

In one embodiment, the database 250 is configured to store, for the user 205, a set of user-specific parameters 260. The set of user-specific parameters 260 may be used to model the glucoregulatory system of the user 205. The set of user-specific parameter 260 includes one or more of: patient age, patient weight, endogenous glucose production, noninsulin-dependent glucose flux, activation rate for insulin remote action, patient insulin sensitivity (e.g. insulin sensitivity of glucose transport, insulin sensitivity of glucose disposal, insulin sensitivity of suppression of EGP), insulin absorption rate, insulin elimination rate, time-to-maximum of CHO absorption, insulin distribution volume, patient daily total dose, patient basal insulin, patient carbohydrate ratios, patient diet and glucose distribution volume.

The database 250 is configured to store, for the user 205, glucose measurements 262. In one embodiment, the glucose measurements 262 are received from the CGM 230. The glucose measurements 262 include, as a non-limiting example, interstitial glucose concentration. As a non-limiting example a rate of glucose appearance from meals could be calculated based on the glucose measurements 262.

The database 250 is configured to store, for the user 205, insulin measurements 264. In one embodiment, the delivered insulin measurements 264 are received from the insulin infusion pump 240. The delivered insulin measurements 264 include one or more of: amount of subcutaneous insulin delivered, and amount of insulin pending to be delivered (i.e. pending by request but not yet delivered), amount of subcutaneous insulin failed to be delivered, insulin-on-board, insulin pump failure or error.

The database 250 is configured to store, for the user 205, a consumed meal information 266. The user 205 may log an indication of a consumed meal on his electronic device 100, which may transmit the indication of the consumed meal in the database 250. The consumed meal information 266 may include one or more of: a composition of the meal, a weight of the meal, a composition of the meal, a type of the meal, an amount of proteins in the meal, a fiber amount in the meal, a carbohydrate amount in the meal, or an estimation thereof.

The database 250 is configured to store, for the user 205, for a given period in time, a set of model parameters 270. Generally speaking, the set of model parameters 270 are parameters representing the glucoregulatory system of the user 205. The set of model parameters 270 generally vary in time to adapt to the user 205. How the set of model parameters 270 are determined will be explained in more detail hereinbelow.

The database 250 is configured to store, for the user 205, state estimates 280. Generally speaking, the state estimates 280 represent a state of the diabetic user 205 for given moments in time. The determination of the state estimates 280 is explained in more detail below.

### Meal Detection Procedure

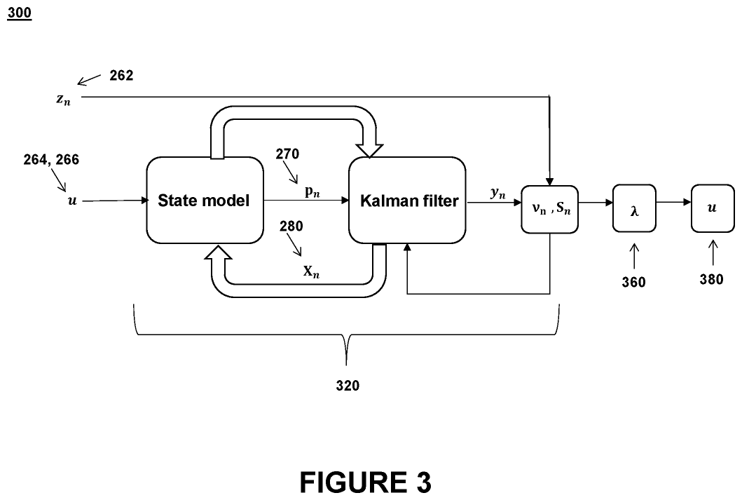

Now turning to FIG. 3, there is depicted a schematic diagram of an unknown meal detection procedure 300 in accordance with a non-limiting embodiment of the present technology.

The unknown meal detection procedure 300 is executed by an electronic device comprising a processor such as the electronic device 100. In one embodiment, the unknown meal detection procedure 300 may be executed by the artificial pancreas system 220 or by another electronic device (not depicted). It is contemplated that the unknown meal detection procedure 300 may be executed by different devices in a distributed manner

In one embodiment, the unknown meal detection procedure 300 is part of the control procedure 245.

The unknown meal detection procedure 300 is adapted to generate a glucoregulatory system model of the user 205 based on historical data of the user 205, predict glucose measurements using the glucoregulatory system model of the user 205, compare the predicted glucose measurement with current glucose measurements, and determine if a meal has not been logged by the user 205. In one embodiment, the unknown meal detection procedure 300 transmits an indication of a missed bolus to the artificial pancreas system 220, which could cause the artificial pancreas system 220 to deliver an insulin bolus. In one embodiment, the indication of the missed bolus includes a recommendation of a bolus to be delivered. The unknown meal detection procedure 300 uses a state-space representation of the glucoregulatory system of the user 205.

The unknown meal detection procedure 300 comprises a state-space modeling procedure 320, a probabilistic detection procedure 360 and an insulin bolusing determination procedure 380.

State-Space Modeling Procedure

The purpose of the state-space modeling procedure 320 is to model the glucoregulatory system of the user 205. The state-space modeling procedure 320 generates a mathematical model describing one or more of the absorption of insulin from the subcutaneous tissue, the absorption of carbohydrate from consumed meals, the changes in glucose due to insulin action, and the changes in glucose due to absorbed carbohydrates. The state-space modeling procedure 320 uses Kalman filtering to predict the glucose measurements.

In one embodiment, the model of the glucoregulatory system 205 of the user may be represented by a set of differential equations. In one embodiment, the glucoregulatory system of the user 205 is described using a linear time-invariant model. As a non-limiting example, the Bergman model may be linearized to describe the glucoregulatory system of the user 205.

In one embodiment, an internal state of the model may be represented by:
Amount of subcutaneous insulin delivered;
Concentration of plasma insulin;
Amount of digested meals;
Rate of glucose appearance from meals;
Glucose plasma concentration; and
Interstitial glucose concentration.

In one embodiment, the state-space modeling procedure 320 generates a model having a set of model parameters 270 represented by variables pₙ. The set of model parameters 270 allow representing the observed glucose measurements 262. The state-space modeling procedure 320 uses a state-space representation to determine state estimates 280. The state estimates 280 in a state-space representation are values that evolve through time in a way that depends on the values they have at any given time and also depends on the externally imposed values of input variables. Output variables' values depend on the values of the state estimates.

Kalman filtering is then used to determine if the glucose measurements are explained by the set of model parameters 270, and the delivered insulin measurements 264 and the consumed meal information 266.

A Kalman filter, also known as linear quadratic estimation (LQE), is an algorithm using a series of measurements over time, which may contain noise and/or inaccuracies, to produce estimates of unknown variables, which could be more accurate than those on based a single measurement. In other words, it is a set of equations implementing a predictor-corrector type estimator to minimize an estimated covariance when conditions are respected, where the equations are executed recursively by an electronic device such as the electronic device 100.

The state-space modeling procedure 320 is configured to receive actual glucose measurements. In one embodiment, the state-space modeling procedure 320 receives the actual glucose measurements from the artificial pancreas system 220.

The state-space modeling procedure 320 is configured to receive glucose measurements 262 from the CGM 230 and/or the database 250. The glucose measurements 262 include the N previous glucose measurements zₙ={z_{n-N+1}, . . . , zₙ},

The state-space modeling procedure 320 is configured to receive, from the insulin infusion pump 240 and/or the database 250, the delivered insulin amounts 264. The delivered insulin amounts 264 includes insulin amounts for time n−N.

The state-space modeling procedure 320 is configured to receive, from the database 250, the consumed meal information 266. The consumed meal information 266 includes consumed meals logged by the user for time n−N.

The delivered insulin amounts 264 and the consumed meal information 266 may be represented together as Uₙ={U_{n-N}, . . . , Uₙ₋₁}. It should be noted that any other inputs that could affect glucose levels in the user 205 could be added such as, but not limited to exercise, and heart rate.

In one embodiment, for a state Xₙ of the user 205 at a time n, the set of model parameters 270 represented by pₙ, the state evolves following the state space model:

Xₙ=A(pₙ)Xₙ₋₁+B(pₙ)Uₙ, (1a)

yₙ=C(pₙ)Xₙ, (1b)

where Uₙ are all the inputs to the system: the delivered insulin amounts 264 and the consumed meal information 266 at time n, and (A(pₙ), B(pₙ), C(pₙ)) are a set of state matrix, input matrix, and output matrix, for the set of parameters 270 pₙ.

In one embodiment, a standard linear Kalman filter is represented by the following equations:

\hat{X}_{n|n-1}=A\hat{X}ₙ₋₁+BUₙ₋₁, (2a)

P_{n|n-1}=APₙ₋₁A^{T}+Q, (2b)

Sₙ=CP_{n|n-1}C^{T}+R, (2c)

Kₙ=P_{n|n-1}C^{T}Sₙ⁻¹, (2d)

\hat{X}(\hat{X}_{n|n-1}+Kₙ(vₙ), (2e)

Pₙ=P_{n|n-1}−KₙCP_{n|n-1}, (2f)

where \hat{X} is the state estimate; P is the covariance matrix of the state estimate;

Q is a process noise covariance matrix; R is measurement noise covariance matrix; Kₙ is the Kalman gain;

vₙ=zₙ−yₙ is an innovation parameter indicative of a mismatch between the actual glucose measurement zₙ, and the predicted measurement yₙ=C\hat{X}_{n|n-1} by the state-space modeling procedure 320.

In one embodiment, the innovation parameter can be considered to be or comprise an innovation covariance parameter. In one embodiment, the innovation parameter can be considered to be or comprise a test statistic.

The innovation parameter quantifies by how much the actual glucose measurement zₙ, and the predicted measurement yₙ values differ. In one embodiment, the innovation parameter is proportional to a difference between the actual glucose measurement zₙ, and the predicted measurement yₙ. Thus, the higher the innovation parameter value, the higher the mismatch between the actual glucose measurement zₙ, and the predicted measurement yₙ. Conversely, the lower the innovation parameter value, the lower the mismatch between the actual glucose measurement zₙ, and the predicted measurement yₙ.

It will be appreciated that the innovation parameter indicative of the mismatch (or lack thereof) between the actual glucose measurement zₙ, and the predicted measurement yₙ may be determined in various ways, and corrective factors or thresholds may be used to determine the innovation parameter. In one embodiment, the value of the innovation parameter indicative of a mismatch between the actual glucose measurement zₙ, and the predicted measurement yₙ may be determined based on a threshold, i.e. if a difference between the actual glucose measurement zₙ, and the predicted measurement yₙ is above (or below)_ a threshold, the value of the innovation parameter may be rounded to another value. Thus, values of the actual glucose measurement zₙ, and the predicted measurement yₙ may be considered “equal” if within a given range.

Sₙ is a covariance of the innovation parameter vₙ.

The Kalman filter is said to be consistent when the probability distribution function of the true state Xₙ is Gaussian with mean Xₙ and covariance Pₙ. Thus, the Kalman filter is consistent when the innovation parameter sequence {v₁, . . . , vₙ} is independent and identically distributed (i.i.d.) and follows a zero-mean Gaussian distribution with covariance Sₙ of the innovation parameter. The consistency of a Kalman filter follows from the hypothesis that the process and measurement noises are i.i.d. zero-mean Gaussian with known covariance matrices Q, and R. A change in the process noise, for instance, an external disturbance, may cause the Kalman filter to become inconsistent.

The state-space modeling procedure 320 may determine or receive an a priori distribution of [symbol] (pₙ) of the set of model parameters 270 based on specific characteristics of the patient, e.g. the set of user-specific parameters 260, and common knowledge such as total daily insulin dose, e.g. the delivered insulin amounts 264 for a day.

In the context of the present technology, the state-space modeling procedure 320 adjusts or updates the set of model parameters 270 to fit the most recent glucose trends, i.e. glucose measurements 262 received from the CGM 230 and/or the database 250, insulin measurements 264 received from the insulin infusion pump 240 and/or the database 250, and meal information 266 received from the user 205.

In one embodiment, if X_{n-N} is a known state at time n−N, the state-space modeling procedure 320 determines a sequence of state propagations Xₙ={X_{n-N}, . . . , Xₙ₋₁} by using a model with the set of model parameters 270 pₙ, the state matrices (Aₚ_{ₙ}, Bₚ_{ₙ}, Cₚ_{ₙ}), and known insulin measurements 264 and the consumed meal information 266 Uₙ={U_{n-N}, . . . , Uₙ₋₁}.

In one embodiment, maximum likelihood estimator of the set of parameters 270 pₙ, describing the last N glucose measurements zₙ={z_{n-N+1}, . . . , zₙ} is obtained by maximizing a likelihood function:

pₙ ∈ arg max [symbol] (Zₙ|Xₙ, Uₙ, pₙ). (3)

It is contemplated that other methods may be used, such as recursive least square for example.

The maximum a posteriori probability estimator (MAP) of the set of parameters 270 pₙ is obtained by:

pₙ ∈ arg max [symbol] (Zₙ|Xₙ, Uₙ, pₙ) [symbol] (pₙ) (4)

Assuming that the measurements are mutually conditionally independent when conditioned on their corresponding state and input, the distribution of the glucose measurements 262 given the states, the delivered insulin amounts 264 and the set of parameters 270 may be expressed as:

[symbol]

(Zₙ|Xₙ, Uₙ, pₙ)˜Π_{k=n-N+1}ⁿ [symbol] (zₖ|Xₖ₋₁, Uₖ₋₁, pₙ), (5)

Assuming a zero-mean Gaussian measurement noise with constant covariance r², for k ∈ [n−N, n], the distribution is expressed as:

$$𝒫 ( {z}_{k} \vert {X}_{k - 1} , {U}_{k - 1} , {p}_{n} ) ∼ exp ( \frac{- 1}{2 {r}^{2}} {( {z}_{k} - {C}_{{p}_{n}} ( {A}_{{p}_{n}} {X}_{k - 1} + {B}_{{p}_{n}} {U}_{k - 1} ) )}^{2} ) , \qquad ( 6 )$$

The state-space modeling procedure 320 uses a maximum a posteriori estimation to adjust the set of model parameters 270. A Kalman filter is then executed using glucose measurements 262 (Zₙ), the known insulin measurements 264 and the consumed meal information 266 (Uₙ) and the state at time n−N (X_{n-N}). The set of model parameters 270 are adjusted to fit the most recent observed glucose trend.

In one embodiment, the state-space modeling procedure 320 is configured to execute the following:
At time k, the state-space modeling procedure 320 determines the Kalman state estimate \hat{X}ₙ corresponding to glucose measurement zₙ based on the set of patient parameters 270 represented by pₙ
Every M epochs, the state-space modeling procedure 320 estimates the set of user parameters 270 based on: N glucose measurements from the glucose measurements 262, the delivered insulin amounts 264 and the consumed meal information 266 at time n−N, and a state estimate at time n−N. In one embodiment, the state-space modeling procedure 320 estimates the set of user parameters 270 by a maximum-a-posteriori method:

pₙ ∈ arg min {Σ_{k=n-N+1}ⁿ(yₖ−C\hat{X}ₖ(p))^{T}R_{MAP}⁻¹(ŷₖ−C\hat{X}ₖ(p))+(p−p_{MEAN})^{T}P_{COV}⁻¹(p−p_{MEAN})+\hat{X}_{n-N}^{T}(p_{n-N})P_{n-N}⁻¹\hat{X}_{n-N}(p_{n-N})} (7)

\hat{X}ₖ(p)=A(p)^{k-n+N}\hat{X}_{n-N}+Σᵣ₌₁^{k-n+N}A(p)^{r-1}B(p)U_{n-N+r-1}, (8)
Where p_{MEAN}, P_{COV} are the prior mean and covariance of the distribution of set of user parameters 270 p, R_{MAP} is the covariance of the measurements, P_{n-N} is the covariance of the state estimate X_{n-N}, and \hat{X}ₖ(p) is the state resulting from \hat{X}_{n-N} and model parameter pₖ at time k.
Every M epochs, the state-space modeling procedure 320 executes a Kalman filter from time n−N to current time n, based on the new set of user parameters 270 pₙ.
The state-space modeling procedure 320 propagates the set of model parameters 270, i.e. pₙ=pₙ₋₁ when the meal detection procedure does not estimate the set of model parameters 270 (i.e. when iterations of the Kalman filter do not correspond to the M epochs) and a one-step Kalman filter is applied.

The state-space modeling procedure 320 stores, in the database 250, the set of model parameters 270, and the state estimates 280 at every iteration.

The state-space modeling procedure 320 stores, in the database 250, the innovation parameter vₙ indicative of a mismatch between the actual glucose measurement zₙ and the predicted glucose measurement yₙ, the predicted glucose measurement yₙ, the covariance Sₙ of the innovation parameter vₙ, and the Kalman gain Kₙ. In one embodiment, the values may be obtained from the artificial pancreas system 200.

In one embodiment, the state-space modeling procedure may be executed in the artificial pancreas 220, and the output may be transferred to the unknown meal detection procedure 320 executed by the electronic device 100.

Probabilistic Detection Procedure

The probabilistic detection procedure 360 is executed to determine, based on the state-space modeling procedure 320, if the user 205 has not logged a meal via the electronic device 100, which causes a change in glucose measurements.

The innovation parameter indicative of a mismatch between a glucose measurement and a predicted glucose measurement may have a large value (i.e. compared to other values of the state parameter) which may be caused by an external disturbance to the system.

Since external disturbances may be due to other factors, the probabilistic meal detection procedure 360 uses a hypothesis test may be used to determine if the external disturbance is caused by a meal that has not been logged by the user 205. Two hypotheses are considered:
H₀: No unknown meal was consumed in the last M iterations (Kalman filter is consistent).
H₁: A meal of size m was consumed without informing the system at time p Å [n−M, n] (Kalman filter is inconsistent).

For a complex hypothesis depending on unknown parameters θ (in this case θ=(p, m) a time and a size of a meal unknown by the user 205) a generalized likelihood ratio test (GLRT) can be used. If Θ is the parameter space of θ, the two hypotheses shall satisfy:

H₀:θ ∈ Θ₀, H₁:θ ∈ Θ₁, and Θ₀ ∪ Θ₁=Θ; Θ₀ ∩ Θ₁=Ø. (9)

Where Θ is a discrete set Θ={(p, m)|p ∈ [n−M, n], m ∈ [mₘᵢₙ, mₘᵢₙ+Δm, . . . , mₘₐₓ]}, where mₘᵢₙ, mₘₐₓ are the smallest and largest detectable unknown meal, and Δm is the minimum detectable difference in unknown meals. With those definitions Θ₀=Ø and Θ₁=Ø. In one embodiment, m is equal to the last 60 minutes, Δm=60 mins, mₘᵢₙ=15 g, and mₘₐₓ=90 g.

A generalized likelihood ratio test (GLRT) is used. The GLRT statistic is written as

$$Λ = \frac{max P ( {V}_{θ} \vert {H}_{0} )}{{max}_{θ ∈ Θ} P ( {V}_{θ} \vert {H}_{1} )} \qquad ( 10 )$$

where V_{θ} is a random variable with a probability distribution function depending on θ. In this case, V_{θ} is a random variable representing the process of Kalman filter innovations {vₙ₋ₘ, . . . , vₙ}.

The null hypothesis P(V_{θ}|H₀) where the Kalman filter is consistent, can be expressed as:

$$P ( {V}_{θ} \vert {H}_{0} ) = {\prod }_{k = n - M}^{n} \frac{1}{\sqrt{2 π {S}_{k}}} exp ( - \frac{{v}_{k}^{2}}{2 {S}_{k}} ) \qquad ( 11 )$$

Under the alternative hypothesis P(V_{θ}|H₁) is stated for θ=(p, m) as

$$P ( {V}_{θ} \vert {H}_{1} ) = {\prod }_{k = n - M}^{n} \frac{1}{\sqrt{2 π {S}_{k}}} exp ( - \frac{{v}_{k}^{2}}{2 {S}_{k}} ) {\prod }_{k = p + 1}^{n} \frac{1}{\sqrt{2 π {S}_{k}}} exp ( - \frac{{( {v}_{k} - {u}_{k}^{θ} )}^{2}}{2 {S}_{k}} ) \qquad ( 12 )$$

And for k ∈ [p+1, n],

uₖ^{θ}=C(Πᵣ₌ₚ₊₁^{k-1}A(I−KᵣC))BUₘ (13)

where Uₘ is a column vector with zeros and the value m in the meal input channel, and I is the identity matrix.

When a meal of size m is consumed at time p, the hypothetical correct state predictions \hat{X}* of the Kalman filter (different from the calculated Kalman filter state \hat{X}) would be \hat{X}*_{p+1|p}=\hat{X}_{p+1|p}+BUₘ.

Thus,

$$\begin{matrix}{\hat{X}}_{p + 2 \vert p + 1}^{*} = A {\hat{X}}_{p + 1}^{*} + B {U}_{p + 1} , \\ = A ( {\hat{X}}_{p + 1 \vert p}^{*} + {K}_{p + 1} ( {z}_{p + 1} - C {\hat{X}}_{p + 1 \vert p}^{*} ) ) + B {U}_{p + 1} , \\ = A ( I - {K}_{p + 1} C ) {\hat{X}}_{p + 1 \vert p}^{*} + {AK}_{p + 1} {z}_{p + 1} + {BU}_{p + 1} , \\ = A ( I - {K}_{p + 1} C ) ( {\hat{X}}_{p + 1 \vert p}^{*} + {BU}_{m} ) + {AK}_{p + 1} {z}_{p + 1} + {BU}_{p + 1} , \\ = A ( {\hat{X}}_{p + 1 \vert p} + {K}_{p + 1} ( {z}_{p + 1} - C {\hat{X}}_{p + 1 \vert p} ) ) + {BU}_{p + 1} + A ( I - {K}_{p + 1} C ) {BU}_{m} , \\ = {\hat{X}}_{p + 2 \vert p + 1} + A ( I - {K}_{p + 1} C ) {BU}_{m} ,\end{matrix} \qquad ( 14 )$$

By recursion, for k ∈ [p+1, n],

\hat{X}*_{k|k-1}=\hat{X}_{k|k-1}+C(Πᵣ₌ₚ₊₁^{k-1}A(I−KᵣC))BUₘ. (15)

It follows that the true innovation parameter v*ₖ satisfies, for k ∈ [p+1, n]

v*ₖ=yₖ−C\hat{X}*_{k|k-1}=vₖ−C(Πᵣ₌ₚ₊₁^{k-1}A(I−KᵣC))BUₘ. (16)

Since v*ₖ follows a zero-mean Gaussian distribution with covariance Sₖ, vₖ will follow a Gaussian distribution with the same covariance and either a zero-mean if k ∈ [n−M, p] or a mean of uₖ^{θ=(p,m)}=C(Πᵣ₌ₚ₊₁^{k—1}A(I−KᵣC))BUₘ if k ∈ [p+1, n].

Therefore:

$$P ( {V}_{θ} \vert {H}_{1} ) = {\prod }_{k = n - M}^{p} \frac{1}{\sqrt{2 π {S}_{k}}} exp ( - \frac{{v}_{k}^{2}}{2 {S}_{k}} ) {\prod }_{k = p + 1}^{n} \frac{1}{\sqrt{2 π {S}_{k}}} exp ( - \frac{{( {v}_{k} - {u}_{k}^{θ} )}^{2}}{2 {S}_{k}} ) . \qquad ( 17 )$$

In one embodiment, θ*=(p*, m*) ∈ arg max P(V_{θ=(p,m)}|H₁) is defined in the probabilistic detection procedure 360 as being the most probable time and size of the hypothetical unknown meal.

Since the sampling distribution of Λ is non-trivial, another test statistic is derived from Λ as:

$$λ = {\sum }_{k = {p}^{*} + 1}^{n} \frac{{u}_{k}^{{θ}^{*}}}{{S}_{k}} {v}_{k} , \qquad ( 18 )$$

Under the null hypothesis, λ follows a zero-mean Gaussian distribution with covariance

$${Σ}_{{p}^{*} + 1}^{n} \frac{{u}_{k}^{{θ}^{{*}^{2}}}}{{S}_{k}} .$$

Thus, the probabilistic detection procedure 360 detects a meal with parameters θ* when λ is smaller than a criterion threshold η satisfying P(λ>η|H₀)<α. In one embodiment, α=0.05. It is contemplated that other values of α are possible.

The probabilistic detection procedure 360 transmits the information to the insulin bolusing determination procedure 380.

Insulin Bolusing Determination Procedure

The insulin bolusing determination procedure 380 receives an indication from the probabilistic detection procedure 360 of a possible missed meal.

When a meal is detected by the probabilistic meal detection procedure 360, the insulin bolusing determination procedure 380 determines a meal size m* and time p* as θ*=(p*, m*) ∈ arg max P(V_{θ=(p,m)}|H₁).

The insulin bolusing determination procedure 38 is configured to execute another Kalman filter routine with the new information about the meal m*. A new state is obtained that contains a better estimation of the patient state. In one embodiment, if m̅ is an estimation of the remaining non-digested meal in the new patient state, the patient safety m̅ may be capped to a give n value such as 20 g.

The insulin bolusing determination procedure 380 determines an insulin bolus, where the insulin bolus u is proportional to the remaining meal, patient carbohydrate ratio CR, glucose level G, glucose target G_{target}, patient-specific correction factor CF and the remaining insulin-on-board (IOB). The insulin bolus may be expressed as:

$$u = \frac{\overline{m}}{CR} + \frac{G - {G}_{target}}{CF} - IOB \qquad ( 19 )$$

The insulin bolusing determination procedure 380 transmits an indication of the insulin bolus to the insulin infusion pump 240, which causes the insulin infusion pump 240 to inject the insulin bolus u. In one embodiment, the insulin bolusing determination procedure 380 transmits an indication of the insulin bolus for display to the user (as an example as a notification on the electronic device 100) who may take appropriate action.

### Method Description

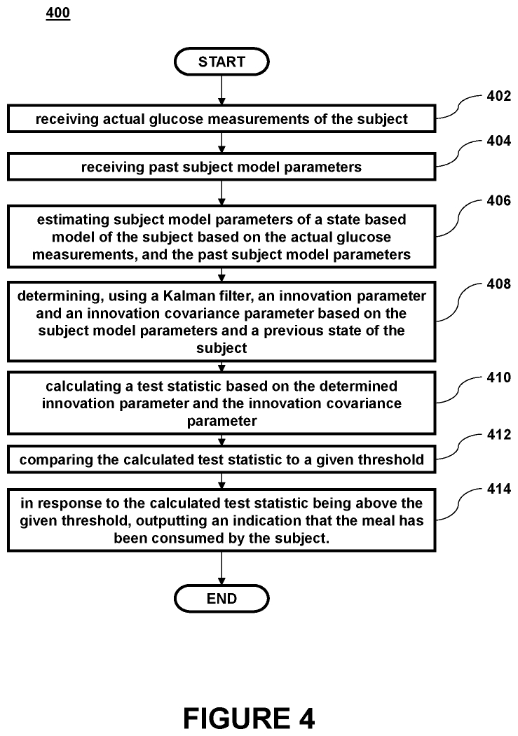

FIG. 4 depicts a flowchart of a method 400 for determining a glucose change in a subject according to non-limiting embodiments of the present technology.

In one embodiment, the method 400 is executed by an electronic device comprising a processor operatively connected to a non-transitory storage medium, such as the electronic device 100.

In one embodiment, the solid-state drive 120 stores computer-readable instructions suitable for being loaded into the random-access memory 130 and executed by the processor 110 and/or the GPU 111 of the electronic device 100. The processor 110, upon executing the computer-readable instructions, is configured or operable to execute the method 400.

The method 400 begins at step 402.

At step 402, the electronic device 100 receives actual glucose measurements of the subject, i.e. the diabetic user 205. In one embodiment, actual glucose measurements are received from the CGM 230. In other embodiments, the actual glucose measurements may be stored in another non-transitory storage medium or received from another electronic device (not depicted)

At step 404, the processor 110 receives past subject model parameters. In one embodiment the past subject model parameters are the set of model parameters 270, which are parameters representing the glucoregulatory system of the user 205.

At step 406, the processor 110 estimates subject model parameters of a state based model of the subject based on: the actual glucose measurements, and the past subject model parameters. In one embodiment, the electronic device 100 determines predicted glucose measurements based on the estimated subject model parameters. In another embodiment, steps 402 to 406 may be replaced by a single step of receiving subject model parameters, where the subject model parameters may have been determined by another electronic device (not depicted).

At step 408, the processor 110 determines, using a Kalman filter, an innovation parameter and an innovation covariance parameter based on the subject model parameters and a previous state of the subject. In one embodiment, the innovation parameter is indicative of a mismatch between the actual glucose measurement zₙ, and the predicted measurement yₙ=C\hat{X}_{n|n-1} by the state based model.

At step 410, the processor 110 calculates a test statistic based on the determined innovation parameter and the innovation covariance parameter. In one embodiment the test statistic is calculated using equation (18).

At step 412, the processor 110 compares the calculated test statistic to a given threshold. In one embodiment, the given threshold has been predetermined based on a number of false positives.

At step 414, the processor 110 outputs an indication that the meal has been consumed by the subject in response to the calculated test statistic being above the given threshold. In one embodiment, the electronic device 100 calculates a value of bolus based on the calculated test statistic and transmits the value of the bolus to an artificial pancreas system.

The method 400 ends.

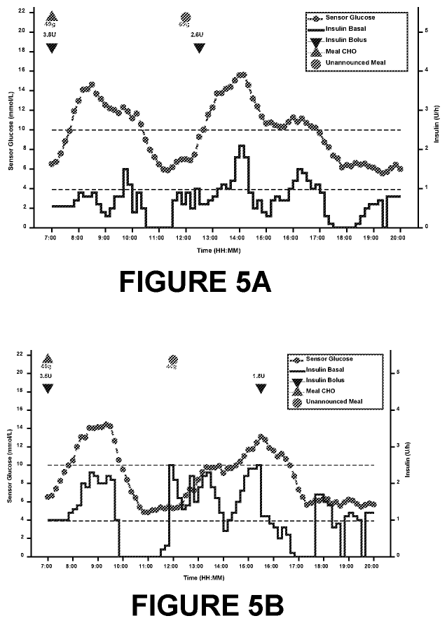

Now turning to FIG. 5 to FIG. 8, there are depicted a plurality of plots of simulations and clinical data experiments.

Simulation Validation

a simulation experiment has been conducted in the purpose of:
Computing the sensitivity of the meal detection procedure, that is the number of detected unknown meals over the total number of unknown meals.
Computing the false alarm rate, that is the number of times the algorithm detects a meal when there was no meal consumed.
Evaluating the effects of introducing a meal detection procedure alongside a traditional closed-loop insulin dosing algorithm on overall glycemic control.

Simulation Setup

The glucoregulatory system of T1D patients is nonlinear and time-varying. To simulate patients' intra- and inter-variability a simulation model presented by Wilinska et al. with time-varying parameters is implemented. To account for variability between patients, model parameters are randomly sampled from a prior distribution. Moreover, the intra-individual variability is accounted for by making some parameters oscillate periodically (with random frequencies and phases) (TABLE I). The simulation is augmented with a correlated noise in glucose measurements (coefficient of variation 7% and correlation of 80%).

TABLE I
Hovorka's model parameters used to sample virtual patients

| | | Inter- |
|---|---|---|
| Parameter description | Intra- variability | variability |
| BW Patient Weight | BW~ [symbol] (65, 95) | Stationary |
| EGP₀ Endogenous glucose production (μmol/(kg min)) | log(EGP₀)~ [symbol] (log(17.0), 0.2) | Oscillatory |
| F₀₁ Noninsulin-dependent glucose flux (μmol/(kg min)) | log(F₀₁)~ [symbol] (log(11.0), 0.1) | Oscillatory |
| k₁₂ Transfer rate from non-accessible (1/min) | log(k₁₂)~ [symbol] (log(0.05), 0.4) | Oscillatory |
| kₐ₁ Activition rate (1/min) | log(kₐ₁)~ [symbol] (log(0.0035), 0.4) | Oscillatory |
| kₐ₂ Activation rate (1/min) | log(kₐ₂)~ [symbol] (log(0.055), 0.4) | Oscillatory |
| kₐ₃ Activation rate (1/min) | log(kₐ₃)~ [symbol] (log(0.025), 0.4) | Oscillatory |
| Sₜ Insulin sensitivity of glucose transport (L/(min mU)) | log(Sₜ)~ [symbol] (log(18.5e−4), 0.4) | Oscillatory |
| S_{d} Insulin sensitivity of glucose disposal (L/(min mU)) | log(S_{d})~ [symbol] (log(5.1e⁻⁴), 0.4) | Oscillatory |
| Sₑ Insulin sensitivity of suppression of EGP (L/mU) | log(Sₑ)~ [symbol] (log(190e⁻⁴), 0.4) | Oscillatory |
| kₐ Insulin absorption rate (1/min) | log(kₐ)~ [symbol] (log(0.018), 0.3) | Oscillatory |
| kₑ Insulin elimination rate (1/min) | log(kₑ)~ [symbol] (log(0.12), 0.2) | Oscillatory |
| τₘ Time-to-maximum of CHO absorption (min) | $log ( \frac{1}{{τ}_{m}} ) ∼ ( log ( \frac{1}{40} ) , 0.2 )$ | Meal Specific |
| Vᵢ Insulin distribution volume (mL/kg) | log(Vᵢ)~ [symbol] (log(120), 0.1) | Stationary |
| V_{g} Glucose distribution volume (mL/kg). | log(V_{g})~ [symbol] (log(150), 0.1) | Stationary |

A simulation experiment, referred to as “CL+MD”, using 512 virtual patients randomly sampled from the distribution in (TABLE I) is conducted. The meal detection procedure is implemented alongside a closed-loop using a model predictive controller (MPC). The simulation experiment (FIG. 5A) consists of a 13 hours simulation where a virtual patient consumes a breakfast of 40 g carbohydrates (CHO) at 7 am, and a lunch at noon consisting of either a 40 g, 60 g or 80 g CHO.

The morning breakfast is entered into the dosing algorithm and a meal-accompanying bolus is given at breakfast. The lunch is given to the virtual patient but not announced to the insulin dosing algorithm Since the effects of the unknown meal and any given bolus by the meal detection procedure are investigated, no meal is consumed after the lunch meal. A rescue CHO of 15 g is given to the virtual patient when the plasma glucose is below 2.7 mmol/L.

1536 simulations (3 meal sizes×512 virtual patients) where the lunch meal is not announced to the dosing algorithm were conducted. A true positive (TP) is counted when the meal detection procedure successfully flags a meal within 120 minutes of the lunch meal. A false negative (FN) is counted when no meal is flagged by the algorithm within 120 minutes of the lunch meal. The sensitivity is the ratio of TP over the total number of unknown meals. The sensitivity of the meal detection procedure for all meals combined (40 g, 60 g, and 80 g) is 93.23%. Other statistics can be found in TABLE II. Since the detection procedure is driven by glucose increase, it is expected to observe that the sensitivity of the algorithm decreases with the meal size (the smallest sensitivity being for 40 g meals). For unknown moderate meals of 60 g CHO, they are detected 96.29% of the times. In average, the algorithm detects a meal after a jump of glucose values above a threshold of 2.6±1.2 mmol/L, and the detection time of the unknown meal is around 40 minutes. Those values appear to be reasonable to ascertain the meal effects from the glucose increases. Similar values for detection time were observed in other studies.

TABLE II
Performance metrics of the meal detection procedure

| | |
|---|---|
| Sensitivity TP/(TP + FN) | 93.23 % |
| Meal CHO = 40 g | 84.77 % |
| Meal CHO = 60 g | 96.29 % |
| Meal CHO = 80 g | 98.63 % |
| Number of false positives | 64 (4.17 % of 1563) |
| Meal CHO = 40 g | 34 (6.64 % of 512) |
| Meal CHO = 60 g | 16 (3.13 % of 512) |
| Meal CHO = 80 g | 14 (2.73 % of 512) |
| Detection time | 40 [30-50] min |
| Meal CHO = 40 g | 50 [40-60] min |
| Meal CHO = 60 g | 40 [30-50] min |
| Meal CHO = 80 g | 30 [30-40] min |
| Glucose increase at detection time | 2.6 + 1.2 mmol/L |
| Meal CHO = 40 g | 2.4 + 1.5 mmol/L |
| Meal CHO = 60 g | 2.7 + 1.1 mmol/L |
| Meal CHO = 80 g | 2.8 + 1.0 mmol/L |
| Glucose increase 10 min before detection time | 1.4 + 1.0 mmol/L |
| Meal CHO = 40 g | 1.5 + 1.3 mmol/L |
| Meal CHO = 60 g | 1.4 + 0.9 mmol/L |
| Meal CHO = 80 g | 1.2 + 0.7 mmol/L |

A false positive (FP) is when meal detection is made in absence of an unknown meal. In the 19968 hours of simulation (13 hours×1536 simulations), 64 FP were encountered, which represents an FP rate of 4.17% per simulation. The relatively high rate of FP after a 40 g meal (34 out of 64 false positives) was mostly due to the late detection of the unknown meal (after the 120 min threshold), because of small glucose increase. The FP count is 18 (instead of 34) if 180 min are considered instead. FIG. 5B shows a case where an FP detection occurred after a late glucose increase. The delivered bolus was safe and did not cause a hypoglycemia.

Effects on Glycemic Control

Since a classification algorithm is susceptible to flag an FP, it is important to assess the impact of such an event. Also, benefits need to be investigated, on glucose control, of adding a meal detection procedure to a closed-loop system. Two other simulation experiments were thus conducted to answer these two questions. Both experiments had the same structure as the CL+MD experiment: 1536 simulations (3 meal sizes×512 virtual patients) were conducted, where a virtual patient uses a closed-loop algorithm and consumes two meals, a breakfast meal and a lunch meal. However, in both experiments, the closed-loop algorithm only consisted of an MPC without a meal detection procedure.

The first experiment, referred to as “CL+B”, simulates the scenario where the lunch was announced and bolused. The second experiment, referred to as “CL”, simulates the scenario where the lunch was not announced, and the MPC only reacted to the change in glucose levels. The two experiments serve to set base values of expected time spent in hypoglycemia and time spent in hyperglycemia.

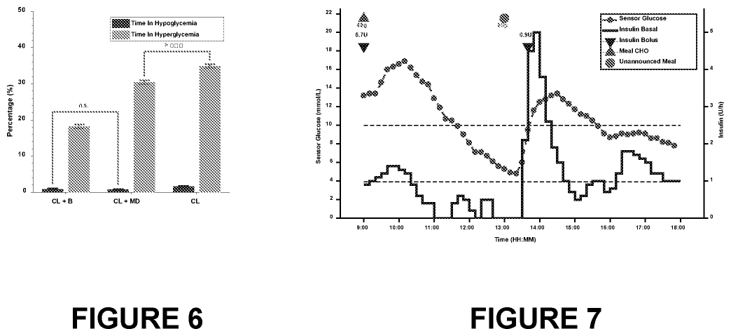

FIG. 6 shows a significant improvement in time spent in hyperglycemia from 34.9% to 30.4% when a meal detection procedure is added to the closed-loop algorithm, which validates the efficacy of the proposed meal detection procedure. TABLE III compares in more details the incremental area under the curve (AUC) in the three experiments for different meals. In average, the AUC is improved by 19% from CL to CL+MD (baseline is CL+B).

TABLE III
Incremental AUC for different meals in all experiments

| AUC (h mmol/L) | CL + B | CL+ MD | CL |
|---|---|---|---|
| CHO = 40 g | 8.8 ± 4.7 | 12.1 ± 4.4 | 13.7 ± 4.9 |
| CHO = 60 g | 11.7 ± 5.3 | 17.0 ± 5.0 | 19.3 ± 5.4 |
| CHO = 80 g | 14.1 ± 6.0 | 21.7 ± 5.9 | 24.4 ± 6.4 |

The meal detection procedure (CL+MD) is safe since no increase in hypoglycemia was observed (FIG. 6) compared to when the exact bolus was delivered (CL+B). To further investigate the safety of the meal detection procedure when an FP is flagged, and an unnecessary bolus is delivered, the time spent in hypoglycemia between simulations where an FP was flagged (n=64) were compared, and simulations where there was no FP (n=1472). The time spent in hypoglycemia when an FP is flagged (1.1±0.35%) has been found non-significantly (p=0.38) different from the time spent in hypoglycemia (0.76±0.08%) when there was no FP. This suggests that there is no apparent correlation between detecting an FP and causing a hypoglycemia with the developed algorithm. The safety of the algorithm after an FP results from the manner the delivered insulin bolus after a meal is flagged was calculated. The computed bolus is a combination of a term that brings glucose levels back to the target ((G−G_{target})/CF−IOB), and a term to cover the detected consumed meal m̅/CR. Since the remaining meal size m̅ is capped to a small CHO value (20 g in this case), the risk of overdosing insulin is minimized. This dosing strategy was found to be the best compromise between not inducing additional hypoglycemia events and decreasing the time spent in hyperglycemia.

Clinical Validation

Experiment Description

Present preliminary results from an ongoing clinical study that assesses the safety and efficacy of closed-loop insulin delivery with and without meal detection module and conventional pump therapy after a missed bolus in adolescents with T1D in inpatient settings are presented. The study consisted of three randomized interventions per patient. Each patient consumed a breakfast with an insulin bolus. Four hours after breakfast, a 60 g lunch was given to the patients without a bolus. Depending on the intervention, insulin doses were based on either a closed-loop algorithm, a closed-loop algorithm with a meal detection module, or the patients' conventional pump therapy. The interventions ended 6 hours after lunch. FIG. 7 shows data from an intervention where the meal detection procedure has been used.

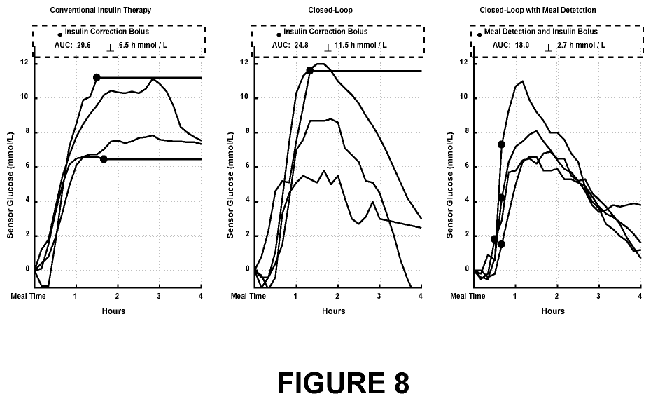

For patients' safety, if their glucose levels were sustained above 18 mmol/L, a correction bolus was delivered. When this happens, glucose levels are assumed to have stayed constant until the end of the intervention. FIG. 8 shows the incremental AUC of four patients who completed all interventions. A trend showing that the meal detection procedure may reduce the incremental AUC after a missed bolus has been observed. In fact, AUC was decreased by 39% with the meal detection procedure compared to 16% without meal detection (baseline is conventional insulin therapy).

To further investigate the meal detection procedure, 108 hours (4 patients×3 visits×9 hours) of clinical data were used to run the meal detection procedure offline. All the 12 unknown meals were detected successfully, and no FP was flagged. The time of meal detection is 35 minutes. Glucose increase at meal detection time is 2.89±1.72 mmol/L and glucose increase 10 minutes before meal detection is 0.45±0.73 mmol/L.

While the present technology has been described in connection with an artificial pancreas system, it is contemplated that the present technology may be used to notify the user of the forgotten insulin and recommend a particular dosage. The user can then take an action, such as delivering the forgotten insulin to himself or herself. In another application, users of conventional pump therapy or multiple daily injections could be reminded if they eat a meal and forget to provide a bolus.

The present technology may also be used to detect disturbances that raise glucose values, such as infusion set failure, or missed meals. The present technology could be used online or offline, to analyze and model data, verify algorithm performance, and as a non-limiting example to identify unknown meals and hypoglycemia treatment.

It should be expressly understood that not all technical effects mentioned herein need to be enjoyed in each and every embodiment of the present technology. For example, embodiments of the present technology may be implemented without the user enjoying some of these technical effects, while other non-limiting embodiments may be implemented with the user enjoying other technical effects or none at all.

Some of these steps and signal sending-receiving are well known in the art and, as such, have been omitted in certain portions of this description for the sake of simplicity. The signals can be sent-received using optical means (such as a fiber-optic connection), electronic means (such as using wired or wireless connection), and mechanical means (such as pressure-based, temperature based or any other suitable physical parameter based).

Modifications and improvements to the above-described implementations of the present technology may become apparent to those skilled in the art. The foregoing description is intended to be exemplary rather than limiting.

1. A computer-implemented method for determining a glucose change in a subject, the method being executable by an electronic device, the method comprising:
receiving subject model parameters of a state-based model of the subject;
determining, using a Kalman filter, an innovation parameter and an innovation covariance parameter based on the subject model parameters and a previous state of the subject;
calculating a test statistic based on the determined innovation parameter and the innovation covariance parameter;
comparing the calculated test statistic to a given threshold; and
in response to the calculated test statistic being above the given threshold, outputting an indication of the glucose change.

2. The method of claim 1,
further comprising, prior to said receiving the subject model parameters:
receiving, by the electronic device, actual glucose measurements of the subject; and
receiving past subject model parameters; and wherein
said receiving the subject model parameters of a state-based model of the subject comprises estimating the subject model parameters based on: the actual glucose measurements, and the past subject model parameters

3. The method of claim 1, further comprising transmitting the indication to at least one of: a display-interface of the electronic device and an artificial pancreas system of the subject.

4. The method of claim 1, wherein the test statistic being above the given threshold is indicative of the Kalman filter being inconsistent.

5. The method of claim 2, wherein said estimating the subject model parameters comprises using a maximum posteriori probability (MAP) estimate.

6. The method of claim 2, wherein said estimating the subject model parameters is further based on: previous glucose measurements, previous insulin measurements and previous consumed meals.

7. The method of claim 1, wherein the test statistic being above the given threshold is indicative of the innovation parameter not being: independent and identically distributed with a zero-mean Gaussian distribution with a covariance corresponding to the covariance of the innovation parameter.

8. The method of claim 1, wherein the glucose change is indicative of an unknown meal, the unknown meal not having been logged by the subject.

9. The method of claim 1, wherein the given threshold is based on a predetermined number of false positives.

10. The method of claim 1, further comprising, prior to said receiving the past subject model parameters:
initializing the past subject model parameters based on: a daily total dose, a basal insulin, and a carbohydrate ratio of the subject.

11. The method of claim 2, wherein the actual glucose measurements are received from a glucose sensor connected to the electronic device.

12. The method of claim 8,
further comprising, prior to said transmitting the indication to the at least one of: the display-interface of the electronic device and the artificial pancreas system of the subject:
determining an insulin bolus of the unknown meal not having been logged by the given user based on: a remaining meal, a patient carbohydrate ratio and a glucose level; and wherein
said transmitting the indication comprises transmitting the insulin bolus.

13. The method of claim 12, further comprising, prior to said determining the insulin bolus:
determining, based on the innovation parameter and the innovation covariance parameter, an unknown meal amount and an unknown meal time.

14. The method of claim 8, wherein the calculated test statistic is representative of a cumulative sum of a correlation between the innovation parameter and a glucose change based on the unknown meal amount and the unknown meal time weighted by the innovation covariance parameter.

15. The method of claim 14, wherein the given threshold is determined based on a: given false positive rate for a random variable with a zero-mean Gaussian distribution and covariance proportional to the square of a most probable glucose increase due to a most probable meal amount and meal time weighted by the innovation covariance parameter.

16. A computer-implemented method for detecting meals consumed by a patient, the method being executed by a processor, the method comprising:
determining a mismatch between actual glucose measurements and predicted glucose measurements;
determining a probability that a meal has been consumed based at least in part on the determined mismatch; and
in response to the determined probability, determining a medication bolus.

17. The method of claim 16, wherein said determining the probability that a meal has been consumed is based, at least in part, on an actual glucose level, a target glucose level, and insulin-on-board.

18. The method of claim 16, further comprising estimating a meal size and a time of consumption of the meal.

19. The method of claim 18, wherein said determining the medication bolus is based, at least in part, on at least one of: the estimated meal size and the estimated time of consumption of the meal.

20. The method of claim 16, wherein said determining that a meal has been consumed is in response to the determined probability breaching a threshold.

21. A system for determining a glucose change in a subject, the system comprising:
a processor;
a non-transitory storage medium operatively connected to the processor, the storage medium comprising computer-readable instructions;
the processor, upon executing the computer-readable instructions, being configured for:
receiving subject model parameters of a state-based model of the subject;
determining, using a Kalman filter, an innovation parameter and an innovation covariance parameter based on the subject model parameters and. a previous state of the subject;
calculating a test statistic based on the determined innovation parameter and the innovation covariance parameter;
comparing the calculated test statistic to a given threshold; and
in response to the calculated test statistic being above the given threshold, outputting an indication of the glucose change.

22. The system of claim 21, wherein
the processor is further configured for, prior to said receiving the subject model parameters:
receiving actual glucose measurements of the subject; and
receiving past subject model parameters; and wherein
said receiving the subject model parameters of a state-based model of the subject comprises estimating the subject model parameters based on: the actual glucose measurements and the past subject model parameters

23. The system of claim 21, wherein the processor is further configured for transmitting the indication to at least one of: a display-interface operatively connected to the processor, and an artificial pancreas system of the subject.

24. The system of claim 21, wherein the test statistic being above the given threshold is indicative of the Kalman filter being inconsistent.

25. The system of claim 22, wherein said
estimating the subject model parameters comprises using a maximum posteriori probability (MAP) estimate.

26. The system of claim 22, wherein said estimating is further based on: previous glucose measurements, previous insulin measurements and previous consumed meals.

27. The system of claim 21, wherein the test statistic being above the given threshold is indicative of the innovation parameter not being: independent and identically distributed with a zero-mean Gaussian distribution with a covariance corresponding to the covariance of the innovation parameter.

28. The system of claim 21, wherein the glucose change is indicative of an unknown meal, the unknown meal not having been logged by the subject.

29. The system of claim 21, wherein the given threshold is based on a predetermined number of false positives.

30. The system of claim 21, wherein the processor is further configured for, prior to said receiving the past subject model parameters:
initializing the past subject model parameters based on: a daily total dose, a basal insulin and a carbohydrate ratio of the subject.

31. The system of claim 22, wherein the actual glucose measurements are received from a glucose sensor connected to the processor.

32. The system of claim 28, wherein
the processor is further configured for, prior to said transmitting the indication to the at least one of: the display-interface operatively connected to the processor and the artificial pancreas system of the subject:
determining an insulin bolus of the unknown meal not having been logged by the given user based on: a remaining meal, a patient carbohydrate ratio and a glucose level; and wherein
said transmitting the indication comprises transmitting the insulin bolus.

33. The system of claim 32, wherein the processor is further configured for, prior to said determining the insulin bolus:
a determining, based on the innovation parameter and the innovation covariance parameter, an unknown meal amount and an unknown meal time.

34. The system of claim 28, wherein the test statistic is representative of a cumulative sum of a correlation between the innovation parameter and a glucose change based on the unknown meal amount and the unknown meal time weighted by the innovation covariance parameter.

35. The system of claim 34, wherein the given threshold is determined based on a: given false positive rate for a random variable with a zero-mean Gaussian distribution and covariance proportional to the square of a most probable glucose increase due to a most probable meal amount and meal time weighted by the innovation covariance parameter.
